(19) United States
(12) Patent Application Publication
(10) Pub. No.: US 2004/0249282 A1
(43) Pub. Date: Dec. 9, 2004

(54) System and method for extracting information based on ultrasound-located landmarks

(76) Inventor: Bjorn Olstad, Stathelle (NO)

Correspondence Address: MCANDREWS HELD & MALLOY, LTD, 500 WEST MADISON STREET, SUITE 3400, CHICAGO, IL, 60661

(21) Appl. No.: 10/849,374

(22) Filed: May 19, 2004

Related U.S. Application Data
(60) Provisional application No. 60/477,434, filed on Jun. 9, 2003.

(51) Int. Cl. A61B 8/00

(52) U.S. Cl. 600/437; 600/450; 600/443

(57) ABSTRACT

An ultrasound machine is disclosed that includes a method and apparatus for generating an image responsive to moving cardiac structure and blood, and for extracting clinically relevant information based on anatomical landmarks located within the heart. At least one processor is responsive to signals received from the heart to locate anatomical landmarks within the cardiac structure and generate position information of the anatomical landmarks, locate clinically relevant locations within the heart based on the position information of the anatomical landmarks, and extract clinically relevant information from the clinically relevant locations within the heart. The landmarks, clinically relevant locations, and clinically relevant information may be displayed to a user of the ultrasound machine.

### RELATED APPLICATIONS/INCORPORATION BY REFERENCE

This application is related to, and claims benefit of and priority from, Provisional Application No. 60/477,434 dated Jun. 9, 2003 (Attorney Docket No. 15-DS-00549 (13109US02) titled “Extracting Information Base on Ultrasound-Located Landmarks”, the complete subject matter of which is incorporated herein by reference in its entirety.

The complete subject matter of each of the following U.S. Patent Applications is incorporated by reference herein in their entirety:

U.S. patent application Ser. No. 10/248,090 filed on Dec. 17, 2002.

U.S. patent application Ser. No. 10/064,032 filed on Jun. 4, 2002.

U.S. patent application Ser. No. 10/064,083 filed on Jun. 10, 2002.

U.S. patent application Ser. No. 10/064,033 filed on Jun. 4, 2002.

U.S. patent application Ser. No. 10/064,084 filed on Jun. 10, 2002.

U.S. patent application Ser. No. 10/064,085 filed on Jun. 10, 2002.

### FEDERALLY SPONSORED RESEARCH OR DEVELOPMENT

[Not Applicable]

### BACKGROUND OF THE INVENTION

Echocardiography is a branch of the ultrasound field that is currently a mixture of subjective image assessment and extraction of key quantitative parameters. Evaluation of cardiac function has been hampered by a lack of well-established parameters that may be used to increase the accuracy and objectivity in the assessment of, for example, coronary artery diseases. It has been shown that inter-observer variability between echo-centers is unacceptably high due to the subjective nature of the cardiac motion assessment.

Much technical and clinical research has focused on the problem and has aimed at defining and validating quantitative parameters. Encouraging clinical validation studies have been reported, which indicate a set of new potential parameters that may be used to increase objectivity and accuracy in the diagnosis of, for instance, coronary artery diseases. Many of the new parameters have been difficult or impossible to assess directly by visual inspection of the ultrasound images generated in real-time. The quantification has typically required a post-processing step with tedious, manual analysis to extract the necessary parameters. Determination of the location of anatomical landmarks in the heart is no exception. Time intensive post-processing techniques or complex, computation-intensive real-time techniques are undesirable.

A method in U.S. Pat. No. 5,601,084 to Sheehan et al. describes imaging and three-dimensionally modeling portions of the heart using imaging data. A method in U.S. Pat. No. 6.099,471 to Torp et al. describes calculating and displaying strain velocity in real time. A method in U.S. Pat. No. 5,515,856 to Olstad et al. describes generating anatomical M-mode displays for investigations of living biological structures, such as heart function, during movement of the structure. A method in U.S. Pat. No. 6,019,724 to Gronningsaeter et al. describes generating quasi-realtime feedback for the purpose of guiding procedures by means of ultrasound imaging.

### BRIEF SUMMARY OF THE INVENTION

An embodiment of the present invention provides an ultrasound system for imaging a heart and extracting clinically relevant information from the heart after having automatically located anatomical landmarks within the heart.

An apparatus is provided in an ultrasound machine for imaging a heart and extracting certain clinically relevant information from the heart based on having previously located certain anatomical landmarks within the heart. In such an environment an apparatus extracting the clinically relevant information comprises a front-end arranged to transmit ultrasound waves into a structure and to generate received signals in response to ultrasound waves backscattered from said structure over a time period.

A processor responsive to the received signals generates a set of analytic parameter values representing movement of the cardiac structure over the time period and analyzes elements of the set of analytic parameter values to automatically extract position information of the anatomical landmarks and track the positions of the landmarks. A processor responsive to the tracked anatomical landmark positions extracts certain clinically relevant information from certain locations within the heart with respect to the tracked anatomical landmarks.

A display is arranged to overlay indicia corresponding to the position information onto an image of the moving structure to indicate to an operator the position of the tracked anatomical landmarks and to display the extracted clinically relevant information.

Certain embodiments of the present invention afford an approach to extract certain clinically relevant information from a heart after automatically locating key anatomical landmarks of the heart, such as the apex and the AV-plane.

A method is also provided in an ultrasound machine for imaging a heart and extracting certain clinically relevant information from the heart based on having previously located certain anatomical landmarks within the heart. In such an environment a method for extracting the clinically relevant information comprises transmitting ultrasound waves into a structure and generating received signals in response to ultrasound waves backscattered from the structure over a time period. A set of analytic parameter values is generated in response to the received signals representing movement of the cardiac structure over the time period. Position information of the anatomical landmarks is automatically extracted and the positions of the landmarks are then tracked. Certain clinically relevant information is extracted from certain locations within the heart with respect to the tracked anatomical landmarks. Indicia corresponding to the position information are overlaid onto the image of the moving structure to indicate to an operator the position of the tracked anatomical landmarks and the extracted clinically relevant information is also displayed.

In one or more embodiments, the at least one clinically relevant location comprises at least one of the lower parts of basal segments of the heart, lower parts of mid segments of the heart, at least one complete myocardial segment of the heart, at least one chamber of the heart, and at least one boundary between at least two chambers of the heart. It is also contemplated that, in at least one or more embodiments, clinically relevant information comprises at least one of Doppler profile information, velocity profile information, strain rate profile information, strain profile information, M-mode information, deformation information, displacement information, and B-mode information. Locating at least one clinically relevant location may comprise performing edge detection of the at least one myocardial segment of the heart to locate endocardium of the at least one myocardial segment.

In one or more embodiments of the method for imaging a heart and extracting certain clinically relevant information from the heart, extracting certain clinically relevant information may comprise presetting at least one M-mode with respect to the at least one anatomical landmark and at least one clinically relevant location. It is further contemplated that extracting may comprise presetting at least one Doppler sample volume with respect to the at least one anatomical landmark and the at least one clinically relevant location. It is also contemplated that extracting may comprise presetting a region-of-interest (ROI) with respect to the at least one anatomical landmark and the at least one clinically relevant location.

In one or more embodiments of the method for imaging a heart and extracting certain clinically relevant information from the heart, the method comprises tracking at least one anatomical landmark in position while locating at least one clinically relevant location and the extracting clinically relevant information. The method may further comprise tracking at least one clinically relevant location in position while extracting clinically relevant information.

### BRIEF DESCRIPTION OF THE DRAWINGS

FIG. 1 is a diagram of an embodiment of an ultrasound machine made in accordance with various aspects of the present invention.

FIG. 2 is a flowchart of an embodiment of a method performed by the machine shown in FIG. 1, in accordance with various aspects of the present invention.

FIG. 3 is a diagram illustrating using the method of FIG. 2 to identify the lower parts of basal segments and mid segments within a heart and to extract clinically relevant information, in accordance with an embodiment of the present invention.

FIG. 4 is a diagram illustrating using the method of FIG. 2 to identify a single myocardial segment or multiple myocardial segments within a heart and to extract clinically relevant information, in accordance with an embodiment of the present invention.

FIG. 5 is a diagram illustrating the relation between strain computed from strain rate imaging and strain visualized and computed from tissue motion imaging, in accordance with an embodiment of the present invention.

FIG. 6 is a diagram illustrating using the method of FIG. 2 to localize a number of short axis anatomical M-modes with respect to anatomical landmarks and to extract clinically relevant information, in accordance with an embodiment of the present invention.

FIG. 7 is a diagram illustrating using the method of FIG. 2 to preset two longitudinal M-modes through two AV-plane locations and to extract clinically relevant information, in accordance with an embodiment of the present invention.

FIG. 8 is a diagram illustrating using the method of FIG. 2 to preset a curved M-mode within a myocardial segment from apex and down to the AV-plane, and to extract clinically relevant information, in accordance with an embodiment of the present invention.

FIG. 9 is a diagram illustrating using the method of FIG. 2 to preset a Doppler sample volume relative to detected anatomical landmarks and to extract clinically relevant information, in accordance with an embodiment of the present invention.

FIG. 10 is a diagram illustrating using the method of FIG. 2 to define a set of points within myocardial segments and to perform edge detection to extract clinically relevant information of the associated endocardium, in accordance with an embodiment of the present invention.

FIG. 11 is a diagram illustrating using the method of FIG. 2 to differentiate between two chambers of a heart and to extract clinically relevant information, in accordance with an embodiment of the present invention.

FIG. 12 is a diagram illustrating using the method of FIG. 2 to tag a display of a heart with a grid and track the grid to extract clinically relevant information, in accordance with an embodiment of the present invention.

The foregoing summary, as well as the following detailed description of certain embodiments of the present invention, will be better understood when read in conjunction with the appended drawings. It should be understood, however, that the present invention is not limited to the arrangements and instrumentality shown in the attached drawings.

### DETAILED DESCRIPTION OF THE INVENTION

An embodiment of the present invention enables the real-time extraction of clinically relevant information within a heart after locating and tracking certain anatomical landmarks of the heart. Moving cardiac structure is monitored to accomplish the function. As used in the specification and claims, structure means non-liquid and non-gas matter, such as cardiac tissue. An embodiment of the present invention helps establish improved, real-time visualization and assessment of certain clinically relevant parameters of the heart. The moving structure is characterized by a set of analytic parameter values corresponding to anatomical points within a myocardial segment of the heart. The set of analytic parameter values may comprise, for example, tissue velocity values, time-integrated tissue velocity values, B-mode tissue intensity values, tissue strain rate values, blood flow values, and mitral valve inferred values.

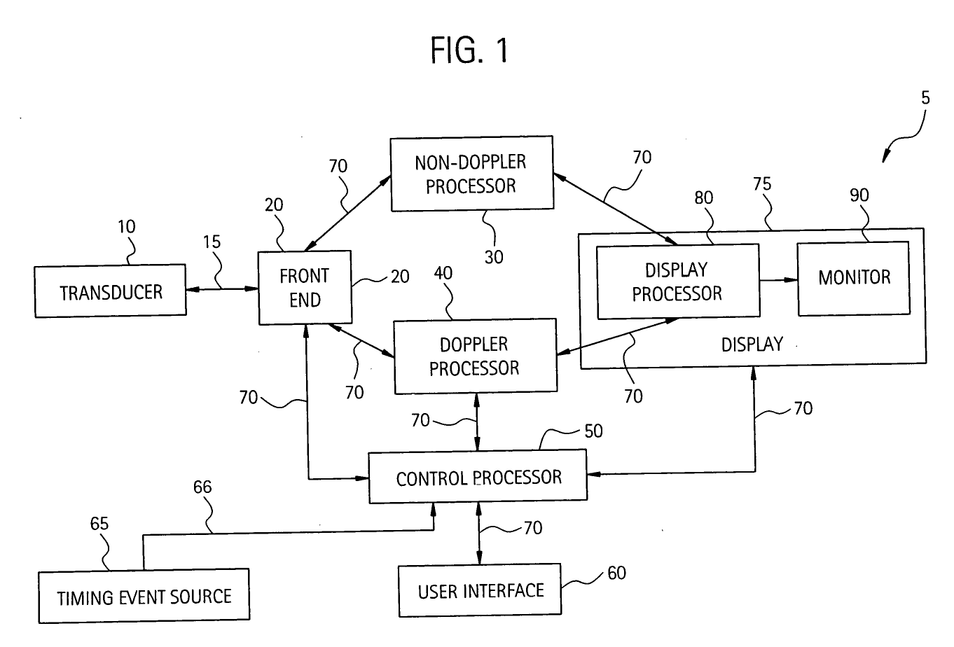

FIG. 1 is a diagram of an embodiment of the present invention comprising an ultrasound machine 5. A transducer 10 is used to transmit ultrasound waves into a subject by converting electrical analog signals to ultrasonic energy and to receive ultrasound waves backscattered from the subject by converting ultrasonic energy to analog electrical signals. A front-end 20 comprising a receiver, transmitter, and beamformer, is used to create the necessary transmitted waveforms, beam patterns, receiver filtering techniques, and demodulation schemes that are used for the various imaging modes. Front-end 20 performs the functions by converting digital data to analog data and vice versa. Front-end 20 interfaces at an analog interface 15 to transducer 10 and interfaces over a digital bus 70 to a non-Doppler processor 30 and a Doppler processor 40 and a control processor 50. Digital bus 70 may comprise several digital sub-buses, each sub-bus having its own unique configuration and providing digital data interfaces to various parts of the ultrasound machine 5.

Non-Doppler processor 30 comprises amplitude detection functions and data compression functions used for imaging modes such as B-mode, B M-mode, and harmonic imaging. Doppler processor 40 comprises clutter filtering functions and movement parameter estimation functions used for imaging modes such as tissue velocity imaging (TVI), strain rate imaging (SRI), and color M-mode. The two processors, 30 and 40, accept digital signal data from the front-end 20, process the digital signal data into estimated parameter values, and pass the estimated parameter values to processor 50 and a display 75 over digital bus 70. The estimated parameter values may be created using the received signals in frequency bands centered at the fundamental, harmonics, or sub-harmonics of the transmitted signals in a manner known to those skilled in the art.

Display 75 comprises scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and passes the analog display signals to a monitor 90. Monitor 90 accepts the analog display signals from display processor 80 and displays the resultant image to the operator on monitor 90.

A user interface 60 allows user commands to be input by the operator to the ultrasound machine 5 through control processor 50. User interface 60 comprises a keyboard, mouse, switches, knobs, buttons, track ball, and on screen menus.

A timing event source 65 is used to generate a cardiac timing event signal 66 that represents the cardiac waveform of the subject. The timing event signal 66 is input to ultrasound machine 5 through control processor 50.

Control processor 50 is the main, central processor of the ultrasound machine 5 and interfaces to various other parts of the ultrasound machine 5 through digital bus 70. Control processor 50 executes the various data algorithms and functions for the various imaging and diagnostic modes. Digital data and commands may be transmitted and received between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

Once certain anatomical landmarks of the heart are identified, (e.g., the AV-planes and apex as described in U.S. patent application Ser. No. 10/248,090 filed on Dec. 17, 2002) certain clinically relevant information may be extracted and displayed to a user of the ultrasound system 5 in accordance with various aspects of the present invention. The various processors of the ultrasound machine 5 described above may be used to extract and display clinically relevant information from various locations within the heart.

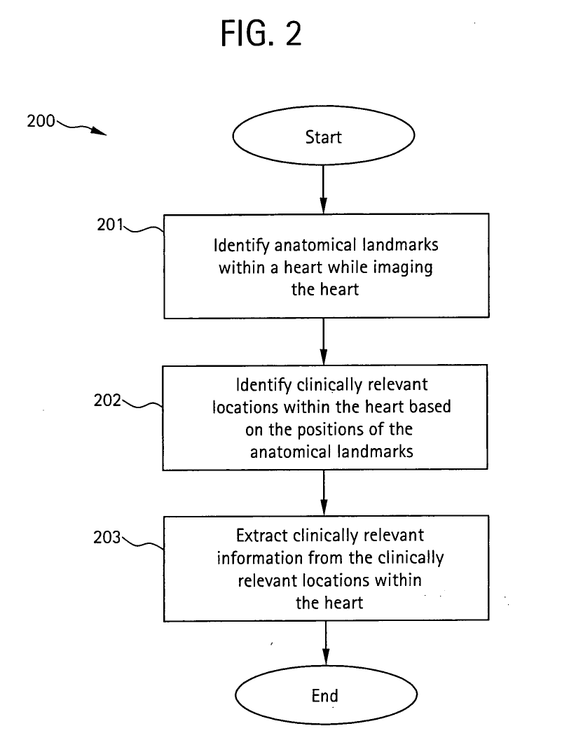

FIG. 2 is a flow chart of an embodiment of a method 200 performed by the machine 5 of FIG. 1 in accordance with various aspects of the present invention. In step 201, anatomical landmarks (e.g., the AV-plane and apex) are identified within the heart while imaging the heart. In step 202, clinically relevant locations are identified based on the positions of the anatomical landmarks. In step 203, clinically relevant information is extracted from the clinically relevant locations within the heart.

As defined herein, clinically relevant information includes at least one of Doppler profile (i.e., over time) information, velocity profile information, strain rate profile information, strain profile information, M-mode information, deformation information, displacement information, and B-mode information.

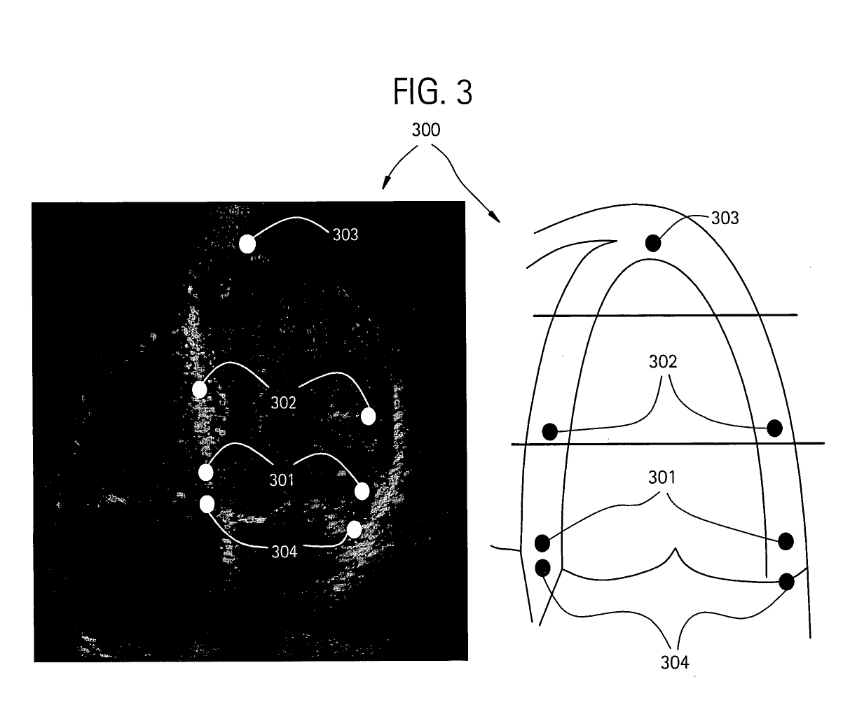

FIG. 3 is a diagram illustrating using the method 200 of FIG. 2 to identify the lower parts of basal segments and mid segments within a heart and to extract clinically relevant information, in accordance with an embodiment of the present invention. Detected landmarks may be used to identify related locations within the heart given by relative positioning and local image characteristics. FIG. 3 shows two depictions of a heart 300. On the left is an image of the heart 300 with various markers overlaying certain anatomical locations. On the right is a graphical illustration of the heart 300 with various markers overlaying certain anatomical locations. FIG. 3 illustrates such an example where the lower parts of myocardium in the basal segments 301 of the heart 300 and the lower part of the mid segments 302 of the heart 300 have been identified relative to the detected landmarks (i.e., apex 303 and AV-plane 304).

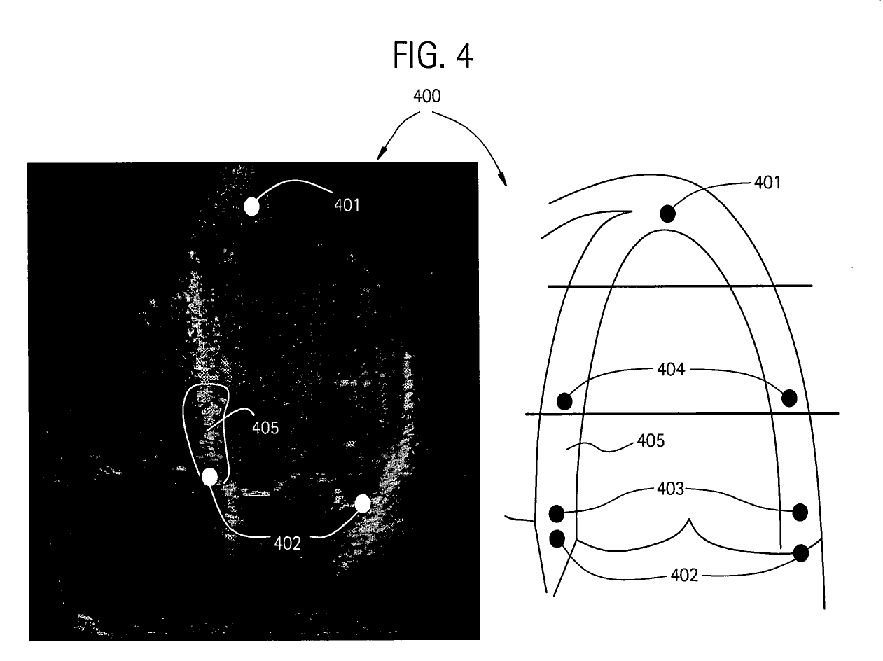

FIG. 4 is a diagram illustrating using the method 200 of FIG. 2 to identify a single myocardial segment or multiple myocardial segments within a heart and to extract clinically relevant information, in accordance with an embodiment of the present invention. FIG. 4 illustrates how locations in the heart 400 such as those shown in FIG. 3, combined with boundary detection, may be used to identify a single myocardial segment 405 or multiple myocardial segments. The locations are marked as apex 401, AV-plane 402, lower part of basal segments 403, and lower part of mid segments 404. The segments defined in the 16-segment model of ASE or similar schemes may be identified. Based on such segmentation, representative clinically relevant parameters may be computed for the segment 405, in accordance with various aspects of the present invention.

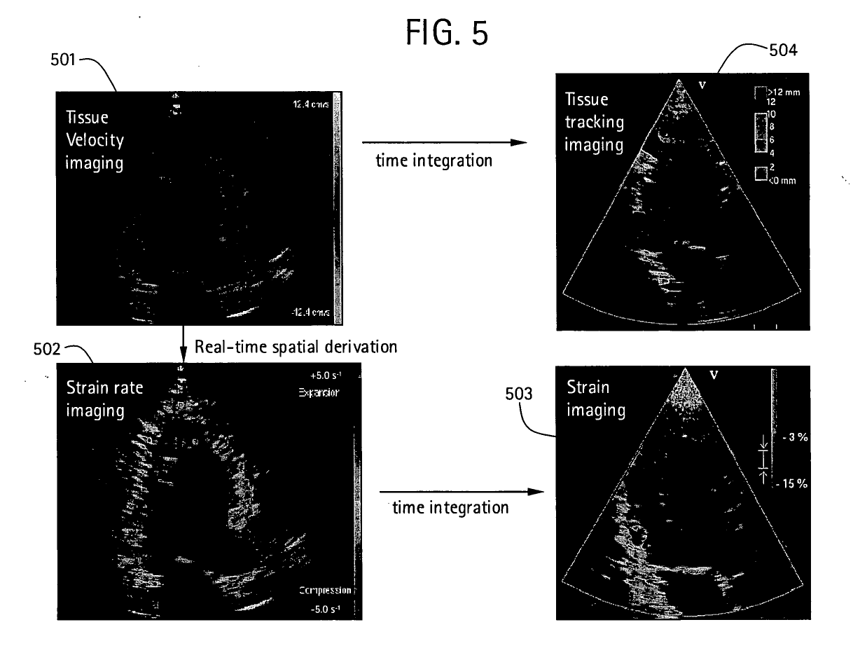

FIG. 5 is a diagram illustrating the relation between strain computed from strain rate imaging and strain visualized and computed from tissue motion imaging, in accordance with an embodiment of the present invention. The upper left of FIG. 5 is a tissue velocity image 501. If the gradient of the tissue velocity is computed along the ultrasound beam, then a strain rate image 502 may be obtained. Such an example is shown in the lower left of FIG. 5. The strain rate values for a given spatial or anatomical location may be combined for a time interval such as systole to compute the local strain as a total deformation in percentage. The lower right of FIG. 5 illustrates such an example where the total systolic strain 503 is used to color encode myocardium. Alternatively, a discrete color encoding 504 of the systolic motion values may be constructed as shown in the upper right corner of FIG. 5. All of these data sources represent possible quantitative clinically relevant information that may be extracted either as simple values or time profiles at locations relative to the detected landmarks.

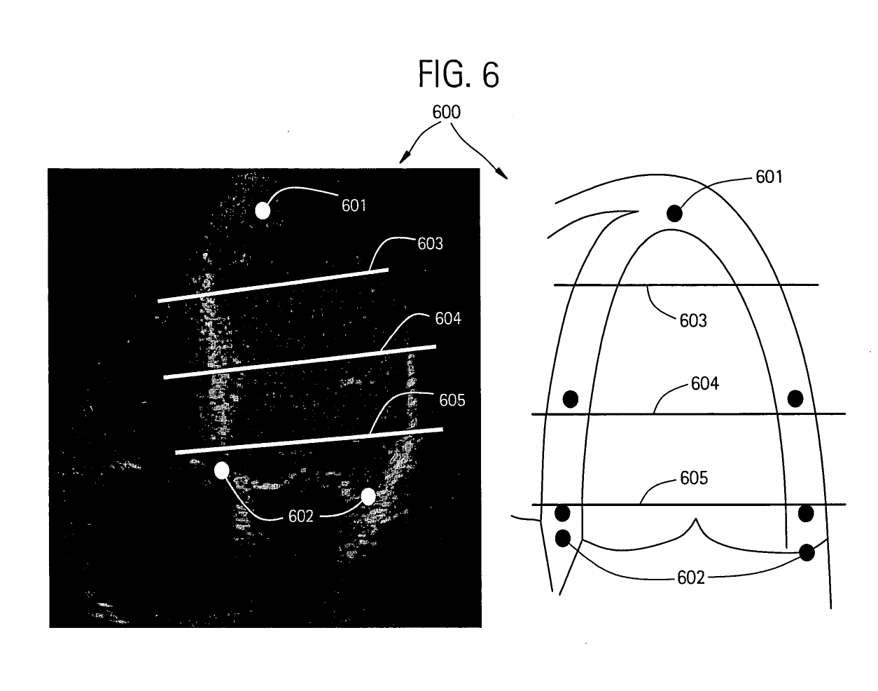

The detected landmarks and related locations may be used to preset the spatial location for acquisition or extraction of additional clinically relevant information. FIG. 6 is a diagram illustrating using the method 200 of FIG. 2 to localize a number of short axis anatomical M-modes with respect to anatomical landmarks and to extract clinically relevant information, in accordance with an embodiment of the present invention. FIG. 6 illustrates how a given number of short axis anatomical M-modes 603, 604, and 605 may be localized as fixed geometrical percentages relative to apex 601 and the two AV-plane locations 602 within a heart 600, in accordance with an embodiment of the present invention.

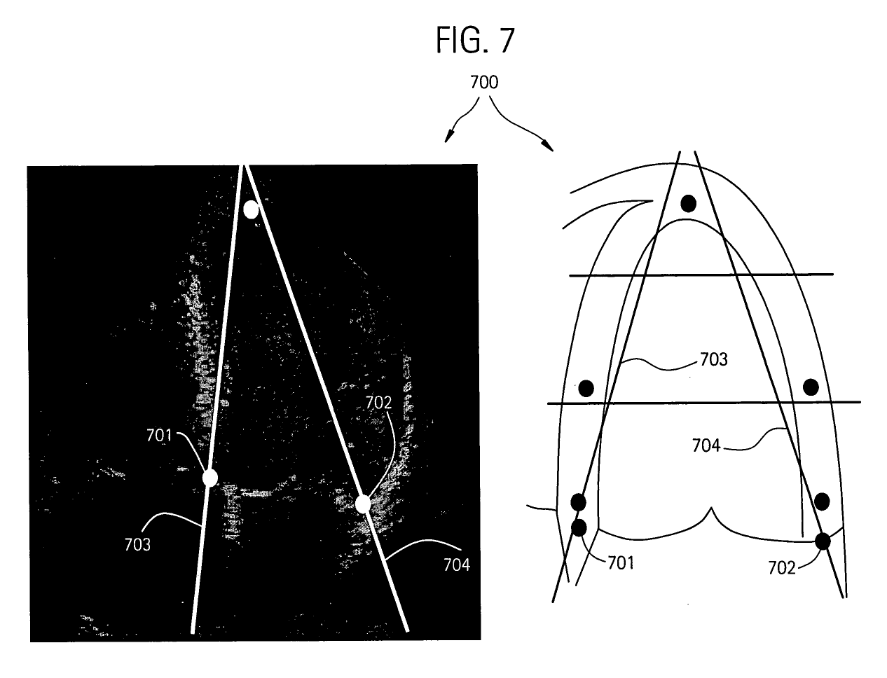

FIG. 7 is a diagram illustrating using the method 200 of FIG. 2 to preset two longitudinal M-modes through two AV-plane locations and to extract clinically relevant information, in accordance with an embodiment of the present invention. FIG. 7 illustrates how two longitudinal M-modes 703 and 704 may be preset through the two AV-plane locations 701 and 702 in order to display the longitudinal AV-motion in two M-modes within the heart 700, in accordance with an embodiment of the present invention.

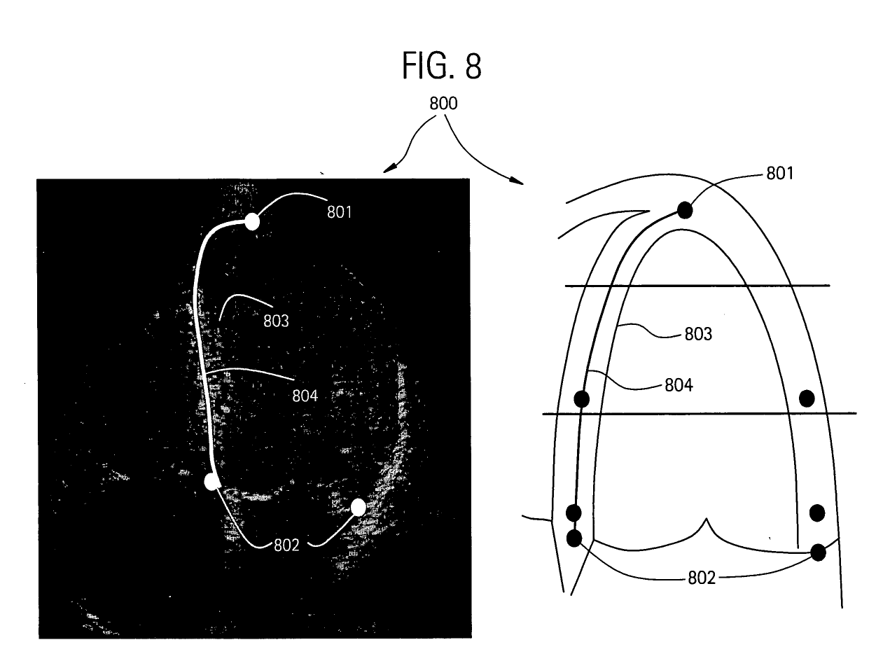

FIG. 8 is a diagram illustrating using the method 200 of FIG. 2 to preset a curved M-mode within a myocardial segment from apex and down to the AV-plane, and to extract clinically relevant information, in accordance with an embodiment of the present invention. FIG. 8 shows how a curved M-mode 804 from apex 801 and down to the AV-plane 802 in the middle of myocardium 803 may be preset by either the landmarks alone or in combination with local image analysis to keep the curve 804 inside myocardium 803 within the heart 800, in accordance with an embodiment of the present invention.

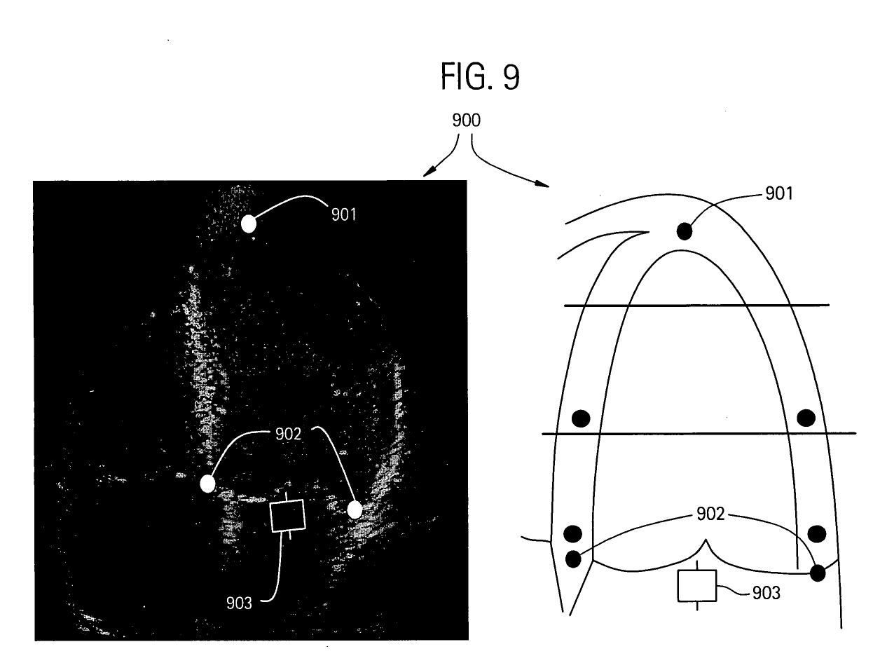

FIG. 9 is a diagram illustrating using the method 200 of FIG. 2 to preset a Doppler sample volume relative to detected anatomical landmarks and to extract clinically relevant information, in accordance with an embodiment of the present invention. FIG. 9 illustrates how a sample volume 903 for Doppler measurements may be preset relative to the detected landmarks 901 (apex) and 902 (AV-plane) within the heart 900. Such a technique may be applied for PW and CW Doppler and for both inspection of blood flow and measurement of myocardial function.

In accordance with an embodiment of the present invention, a region-of-interest (ROI) may be preset with respect to the anatomical landmarks to extract information from the clinically relevant locations. The information extracted may include any of Doppler information over time, velocity information over time, strain rate information over time, strain information over time, M-mode information, deformation information, displacement information, and B-mode information.

The locations for all of the M-modes, curved M-modes, sample volumes, and ROI's may be tracked in order to follow the motion of the locations, in accordance with an embodiment of the present invention. Also, indicia may be overlaid onto the anatomical landmarks and/or the clinically relevant locations to clearly display the positions of the landmarks and/or locations.

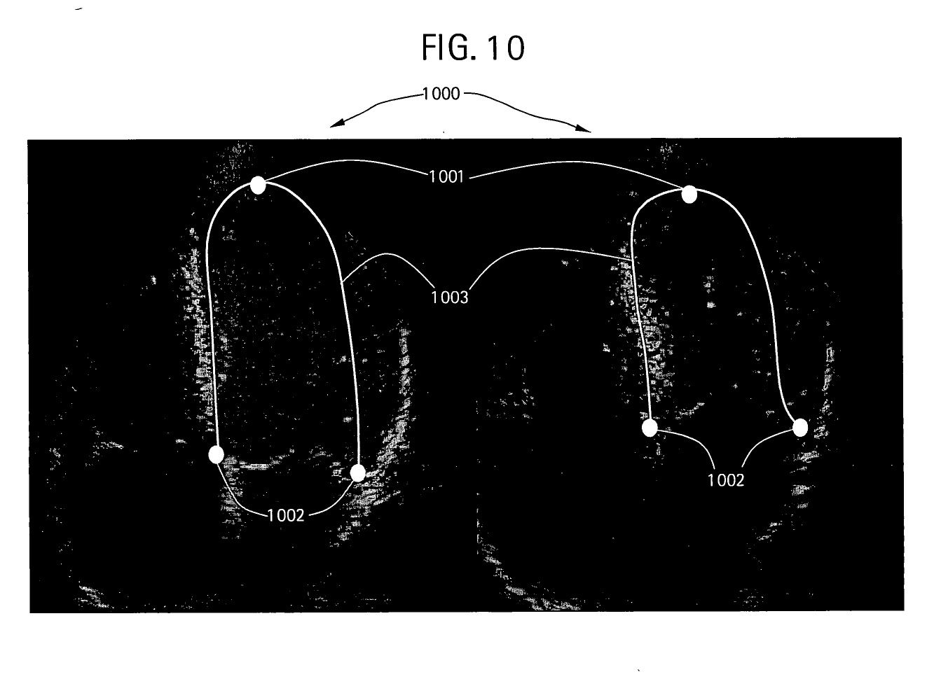

FIG. 10 is a diagram illustrating using the method 200 of FIG. 2 to define a set of points within myocardial segments and to perform edge detection to extract clinically relevant information of the associated endocardium, in accordance with an embodiment of the present invention. Automatic edge detection of endocardium remains a challenging task. FIG. 10 illustrates how the techniques taught herein (i.e., similar to the curved M-mode localization) may be used to either define a good region of interest for the edge detection, or provide an initial estimate that may be used to search for the actual boundary with edge detection algorithms such as active contours. FIG. 10 shows two views of a heart 1000 with the apex 1001 and the AV-plane 1002 identified. A contour 1003, estimating the approximate inside of myocardial segments in the heart 1000 based on the anatomical landmarks, is drawn as the apex and AV-plane locations are tracked. Edge detection of the endocardium may then be performed using edge detection techniques using the contour as a set of starting points.

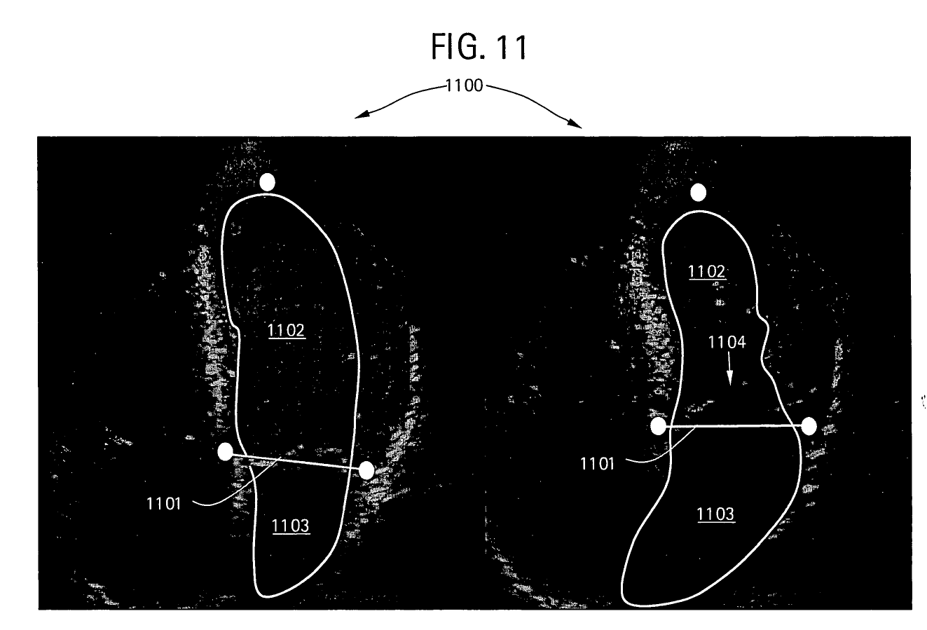

FIG. 11 is a diagram illustrating using the method 200 of FIG. 2 to differentiate between two chambers of a heart and to extract clinically relevant information, in accordance with an embodiment of the present invention. FIG. 11 shows a different application in edge detection within two views of a heart 1100. Even an ideal blood/tissue segmentation may not, at all instances in the cardiac cycle, be able to separate between the ventricle 1102 and the atrium 1103. The two chambers 1102 and 1103 are completely connected with blood in diastole when the mitral valve 1104 is open. Detection of the AV-plane 1101 may be used to separate a blood/tissue segmentation into the ventricle and atrial components.

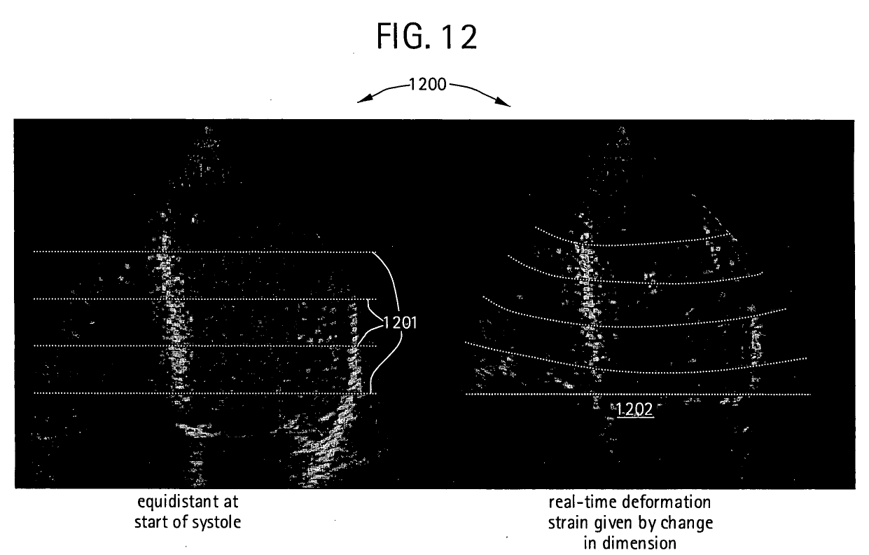

FIG. 12 is a diagram illustrating using the method 200 of FIG. 2 to tag a display of a heart with a grid and track the grid to extract clinically relevant information, in accordance with an embodiment of the present invention. FIG. 12 illustrates a possible scenario for implementation of a tagging display based on tissue tracking. In accordance with an embodiment of the present invention, a time interval relative to the cardiac cycle is selected. The time interval may equal the complete cardiac cycle, for example. At the start of the time interval, a fixed graphical grid 1201 is drawn on top of the ultrasound image 1200. Any shape, including any one or two-dimensional grids may be used. The left hand side of FIG. 12 illustrates a one-dimensional grid 1201 where equidistant horizontal lines are used. Another good candidate is to use an equidistant set of lines with constant depth in the polar geometry representation of the ultrasound image. The anatomical locations are then tracked throughout the selected time interval with either one-dimensional techniques along the ultrasound beam, or two-dimensional techniques.

The right hand side of FIG. 12 illustrates the display of a frame in the selected time interval where the motion and deformation of the original grid pattern 1201 is used to visualize the motion and strain properties. The display mode might be attractive to clinicians because it resembles tagging MR that is used as a gold reference for in-vivo measurements of strain. The detection of landmarks like apex and the AV-plane locations may further enhance the display mode by presetting the grid 1201 relative to the landmarks. Such presetting may assure that a grid line passes through both apex and the AV-plane. The intermediate locations may, for instance, be selected such that the displayed deformations correspond with the appropriate vascular territories. A special grid structure or band 1202 could be added around the AV-plane that corresponds to normal or expected longitudinal motion.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. In an ultrasound machine for generating an image responsive to moving cardiac structure and blood within a heart of a subject, a method comprising:
locating at least one anatomical landmark within the cardiac structure and generating position information of said at least one anatomical landmark;
locating at least one clinically relevant location within the heart based on said position information of said at least one anatomical landmark; and
extracting clinically relevant information from said at least one clinically relevant location within the heart.

2. The method of claim 1 further comprising displaying said clinically relevant information on a display of said ultrasound machine.

3. The method of claim 1 further comprising displaying indicia overlaying said at least one clinically relevant location on a display of said ultrasound machine.

4. The method of claim 1 wherein said at least one anatomical landmark comprises at least one of an apex of the heart and an AV-plane of the heart.

5. The method of claim 1 wherein said at least one clinically relevant location comprises at least one of lower parts of basal segments of the heart, lower parts of mid segments of the heart, at least one complete myocardial segment of the heart, at least one chamber of the heart, and at least one boundary between at least two chambers of the heart.

6. The method of claim 5 wherein said locating at least one clinically relevant location includes performing edge detection of said at least one myocardial segment of the heart to locate endocardium of said at least one myocardial segment.

7. The method of claim 1 wherein said clinically relevant information comprises at least one of Doppler profile information, velocity profile information, strain rate profile information, strain profile information, M-mode information, deformation information, displacement information, and B-mode information.

8. The method of claim 1 wherein said extracting includes presetting at least one M-mode with respect to said at least one anatomical landmark and said at least one clinically relevant location.

9. The method of claim 1 wherein said extracting includes presetting at least one Doppler sample volume with respect to said at least one anatomical landmark and said at least one clinically relevant location.

10. The method of claim 1 wherein said extracting includes presetting a region-of-interest (ROI) with respect to said at least one anatomical landmark and said at least one clinically relevant location.

11. The method of claim 1 further comprising tracking said at least one anatomical landmark in position while performing said locating at least one clinically relevant location and said extracting clinically relevant information.

12. The method of claim 1 further comprising tracking said at least one clinically relevant location in position while performing said extracting clinically relevant information.

13. In an ultrasound machine for generating an image responsive to moving cardiac structure and blood within a heart of a subject, an apparatus comprising:
a front-end arranged to transmit ultrasound waves into the moving cardiac structure and blood and to generate received signals in response to ultrasound waves backscattered from the moving cardiac structure and blood;
at least one processor responsive to said received signals to locate at least one anatomical landmark within the moving cardiac structure and generate position information of said at least one anatomical landmark, locate at least one clinically relevant location within the heart based on said position information of said at least one anatomical landmark, and extract clinically relevant information from said at least one clinically relevant location within the heart.

14. The apparatus of claim 13 further comprising a display processor and monitor to process said position information and display indicia overlaying at least one of said at least one anatomical landmark and said at least one clinically relevant location.

15. The apparatus of claim 13 further comprising a display processor and monitor to process and display said clinically relevant information.

16. The apparatus of claim 13 wherein said at least one anatomical landmark comprises at least one of an apex of the heart and an AV-plane of the heart.

17. The apparatus of claim 13 wherein said at least one clinically relevant location comprises at least one of lower parts of basal segments of the heart, lower parts of mid segments of the heart, at least one complete myocardial segment of the heart, at least one chamber of the heart, and at least one boundary between at least two chambers of the heart.

18. The apparatus of claim 13 wherein said clinically relevant information comprises at least one of Doppler profile information, velocity profile information, strain rate profile information, strain profile information, M-mode information, deformation information, displacement information, and B-mode information.

19. The apparatus of claim 13 wherein said at least one processor comprises at least one of a Doppler processor, a non-Doppler processor, a control processor, and a PC back-end.

20. The apparatus of claim 13 further comprising at least one transducer connected to said front-end to convert electrical signals to said ultrasound waves and vice versa.

21. The apparatus of claim 13 further comprising at least one user interface connecting to said at least one processor to control operation of said ultrasound machine.
